(19) United States
(12) Patent Application Publication
(10) Pub. No.: US 2010/0106111 A1
(43) Pub. Date: Apr. 29, 2010

(54) DISPOSABLE SLEEP MASK FOR TREATING DRY EYE SYNDROME

(75) Inventors: Mary Schwebel, Minneapolis, MN (US); Kays Chinai, Burlington, NJ (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Mary L. Schwebel, Minneapolis, MN (US)

(21) Appl. No.: 12/259,883

(22) Filed: Oct. 28, 2008

(51) Int. Cl. A61F 9/04; B32B 38/00

(52) U.S. Cl. 604/294; 156/245

(57) ABSTRACT

A mask for the relief of dry eye in a dry eye subject, including a unitary shell shaped to cover at least one eye and a portion of a face. The shell includes a first layer and a second layer the first layer that is substantially moisture impermeable and the second layer that is moisture absorbent. The first layer and the second layer meeting at a layer interface. The first layer and the second layer are substantially coextensive over substantially the entire layer interface and are secured together over substantially the entire layer interface.

### FIELD OF THE INVENTION

The present invention generally relates to masks to be worn over the eyes to provide treatment for dry eye syndrome.

### BACKGROUND OF THE INVENTION

Dry eye syndrome is one of the most commonly treated eye problems in the United States. Dry eye syndrome is also known as keratitis sicca, keratoconjunctivitis sicca (KCS) xerophthalmia, and lacrimal insufficiency. It is estimated that over ten million Americans and 30 million persons worldwide suffer from dry eye syndrome.

For a large fraction of dry eye patients, dry eye syndrome creates discomfort or annoyance. For those severely afflicted, dry eye syndrome can be debilitating and, in some circumstances, even sight-threatening. In extremely severe cases, dry eye syndrome can even lead to the loss of an eye.

Dry eye syndrome typically results from deficiency in the quality or quantity of tears produced by the patient. Precomeal tear film has traditionally been considered to have a three-layered structure. The closest to the cornea lies the mucin, or mucus, layer. The mucin layer provides an interface between the corneal epithelium and the remainder of the tear film. Overlying the mucin layer is the watery aqueous layer, which is the thickest layer of the three. The outermost layer of the precorneal tear film is the lipid layer. The lipid layer is an oily film that reduces evaporation from the aqueous layer beneath it.

The middle aqueous layer provides moisture to the corneal tissue, carries important nutrients, and serves to remove metabolic waste produced by the cornea. Deficiency in any of the three layers of the precorneal tear film can result in complaints of dry, gritty feeling or burning eyes.

The mucin that forms the mucin layer, nearest the cornea, is secreted by goblet cells in the conjunctiva. The conjunctiva is the transparent tissue that covers the sclera and the backside of the eyelids. The mucin layer functions to decrease surface tension of the tear film. In addition, the cornea itself is hydrophobic. Without the mucin layer to provide a bridge between the cornea and the aqueous layer, the aqueous layer would bead up and allow dry spot formation on the cornea.

The aqueous layer is secreted primarily by the glands of Wolfring and Krause located in the eyelid margin. The aqueous layer helps provide an optically smooth, transparent surface to the precorneal tear film. The lipid layer is secreted by the meibomian glands, and the glands of Zeiss and Moll. The glands of Zeiss and Moll are also located at the eyelid margin.

Blinking is essential to maintenance of the precomeal tear film. During each blink, the eyelid wipes over the surface of the cornea, smoothing the mucin layer and spreading the overlying aqueous and lipid layers to provide a completely wetted surface. In between blinks, the tear film thins due to evaporation of the aqueous layer. If evaporation is excessive, dry spots may form on the surface of the cornea.

Deficiency, or imperfect quality, of any of the three component layers can lead to dry eye symptoms. Many systemic and external factors can contribute to dry eye syndrome. For example, Sjogren's syndrome is associated with arthritic diseases in combination with dry eye and dry mouth. Deficiency of Vitamin A, use of oral contraceptives and environmental factors can all contribute to dry eye syndrome.

Recent research into the natural history of dry eye syndrome has shown that the disease progresses through four stages. Each stage is a consequence of the preceding stage. The stages are:
1. Loss of water from the aqueous layer of the tear film leading to an increase in the tear film osmolarity;
2. Loss of conjunctival goblet cells and decreased corneal glycogen;
3. Increased loss of corneal squamous epithelial cells;
4. Destabilization of the interface between the corneal surface and the tear film.

Either decreased secretion of tear film components or increased evaporation lead to increased tear film osmolarity and the following stages that lead to eventual corneal decompensation and the serious consequences of dry eye syndrome.

The adnexa of the eye may also be involved in dry eye syndrome. The adnexa of the eye include the structures surrounding the eye such as the eyelids, eye lashes, the tear drainage and tear production structures. Blepharitis commonly contributes to dry eye syndrome. Blepharitis typically results from bacterial infection of the tiny glands in the margin of the eyelid. These glands include the glands of Zeiss, Moll and Wolfring as well as the meibomian glands. Most commonly, the affected glands are the meibomian glands. In bacterial blepharitis, bacterial infection causes the meibomian glands to become plugged, and thus not be able to produce a normal lipid layer to contribute to the tear film. Some bacteria that infect the glands also secrete exotoxins that seep out of the glands into the eye and injure the corneal epithelium.

Treatments of dry eye syndrome vary depending upon the type of presentation. The most common treatment for dry eye syndrome is the use of artificial tear supplements to provide additional moisture and lubrication to the corneal surface. Artificial tear eye drops are placed on the eye by the patient. Artificial tear supplements must be used regularly and often to be effective.

Lubricant ointments may also be employed. Ointments are usually used at bedtime because they tend to be messy and blur vision. For some patients, even the use of ointments is not sufficient to provide comfort during sleep.

Tears drain from the eye through the lacrimal drainage system. Tiny openings at the nasal corner of each upper and lower eyelid are called the lacrimal puncta. The lacrimal puncta lead into ducts that drain into the nasopharynx.

One treatment for dry eye syndrome is to partially or completely close one or more lacrimal puncta to reduce tear outflow into the lacrimal drainage apparatus. Traditionally, this closure was accomplished surgically or by cautery. In the last decade, however, temporary and permanent punctal occlusion plugs have been utilized.

Permanent puncta plugs are typically made from surgical silicone; temporary plugs are generally made of soluble collagen. Collagen plugs dissolve over a period of days and are helpful in diagnosis.

Punctal plugs are placed into the lacrimal puncta, or lacrimal drainage ducts. The plugs impede the outflow of tears from the eye. This approach slows the outflow of tears and retains them in the eyes longer, often relieving symptoms. Punctal plugs have the distinct advantage of being readily removable and avoid the issues of scar formation.

Blepharitis is sometimes treated by the use of antibiotic medications. Another important treatment for blepharitis is the application of warm soaks and lid scrubs. In this form of treatment, the patient applies a warm wet washcloth to the eyelids for a period of time to provide humidity, warmth and to help soften blockage of and restore flow from the meibomian glands. Lid scrubs are practiced by taking a mild, nonirritating soap and vigorously scrubbing the eyelid margins with the eyes closed, so as to massage the meibomian glands and increase production. The surfactant helps to dissolve the greasy blockage of the meibomian glands.

Patients who have severe dry eye syndrome often suffer disrupted sleep because they cannot go for longer than an hour or so without applying tear supplements to the eyes. This can lead to pronounced sleep deprivation and a consequent reduction in quality of life.

A variety of researchers have been seeking other medicinal treatments for dry eye syndrome. Largely, this research is directed at pharmaceutical efforts to increase tear production. In addition it has been discovered that an inflammatory component plays a larger part in dry eye syndrome than was previously thought. Recent research has led to some success in treating the inflammatory component of dry eye syndrome. Restasis® is a topical ophthalmic solution containing the anti-rejection drug Cyclosporin.

Despite the many treatment options available, there remains no cure for dry eye syndrome A great many patients still have substantial and even debilitating discomfort because of dry eye syndrome. Very few treatment options exist to provide comfort for dry eye syndrome patients during sleep.

Sleep masks to treat dry eye syndrome can provide a higher humidity environment in the vicinity of the eyes and adnexa to improve the comfort and health of the eyes. Such masks are disclosed in U.S. Pat. Nos. 7,036,928, 6,886,933, 6,874,884 and 6,641,264 issued to and commonly owned by the inventor named in this application. In addition, U.S. published applications 2006/0157064 and 2005/0022823 also disclose sleep masks to treat dry eye syndrome. Existing masks on the market to ameliorate the effects of dry eye syndrome generally are expensive and relatively complex structures. Dry eye sufferers would benefit from a simple light and comfortable sleep mask. Additionally, dry eye patients would benefit from a sleep mask that is inexpensive and disposable in nature.

### SUMMARY OF THE INVENTION

The dry eye relief mask of the present invention provides an inexpensive and disposable device which can used to increase humidity in the area surrounding the eyes of the dry eye patient during sleep or other times when it is desirable to improve humidity in the local environment of the eyes. Increased humidity reduces evaporation of the tear film, thereby improving comfort and health of the eyes of the dry eye patient.

The dry eye relief mask generally includes a shell having two layers. The shell has a front substantially water impermeable layer, which may be formed of a polymer material and a back water absorbent layer, which may be formed of a polymer or other material. In an example embodiment, the front water impermeable layer and the back water absorbent layer are fused together by heat and pressure in a mold. In another example embodiment, the front water impermeable layer and the back water absorbent layer are bonded together by adhesive and pressure.

In another example embodiment, the shell generally defines a generally planar portion, domes and wall structures. The domes and walls together define eye recesses. In one example embodiment, the eye recesses may take the shape of roughly an irregular pentagon.

In an example embodiment of the invention, absorbent layer 18 may be formed of an open cell hydrophilic foam. In one example embodiment of the invention, the hydrophilic foam may be Medisponge 30W SOO-T or SOS-T produced by Lendell Manufacturing, Inc. Medisponge is a hydrophilic flexible polyurethane foam. In accordance with the invention, an example absorbent layer 18 may have the following properties.

| Property | Specification |
|---|---|
| Pore Structure | Uniform tight pore structure across width |
| Density, lb/ft³ | 5.7 +/− 1 lbs/ft³ |
| Tensile (psi) | 30.2 +/− 5 |
| % Volume Swell | 75% =/− 15% |
| Wet out | less than one minute |
| Saturated Water Retention | 15 +/− 4 times Wt/wt |
| Wrung Water Retention | 1.5 +/− 0.5 |

Substantially impermeable layer 16 in one example embodiment may be formed of a closed cell polyurethane foam. For example, polyethylene foam sheets, such as those that can be obtained from Voltek of Lawrence, Mass. have been found to be suitable for use in the present invention. The following examples illustrate suitable qualities.

### EXAMPLE 1

foam sheet thickness 4.0 mm (Voltek of Lawrence, Mass., Code: LL1077);

density 0.035 grams per cc;

water vapor transmission rate 0.087 g/8 hours/100 sq inches-ASTM E96;

compression set 6% original thickness-ASTM D3575;

shore hardness 18 “A” scale; and

25 % compression load 2.5 psi-ASTM D3575.

Such foam sheet material can be molded into a mask by heat and pressure. In this example the temperature of a mold was maintained at about 120°°C. and the molding time was approximately 2.5 minutes.

### EXAMPLE 2

Polyethylene foam sheets (Voltek Code: LL1036):

Foam sheet thickness 3.4 mm;

Density 0.21 grams cm³;

Water vapor transition rate 0.087 g/8 hours/100 sq inches-ASTM E96;

Compression set 3.8% original thickness;

Shore hardness 30 “A” scale; and

25% compression load 8.5 psi.

A mask produced from this material is less flexible than that described above in Example 1.

### BRIEF DESCRIPTION OF THE DRAWINGS

FIG. 1 is a perspective view of an eye mask in accordance with the invention;

FIG. 2 is a rear plan view of an eye mask as depicted in FIG. 1;

FIG. 3 is cross-sectional view of an eye mask in accordance with the invention taken along section line 3-3 of FIG. 2.

FIG. 4 is a perspective view of another embodiment of an eye mask in accordance with the invention;

FIG. 5 is a rear plan view of the eye mask as depicted in FIG. 4; and

FIG. 6 is a perspective view of partial contents of a kit including an eye mask in accordance with the invention and an eye care solution.

### DETAILED DESCRIPTION

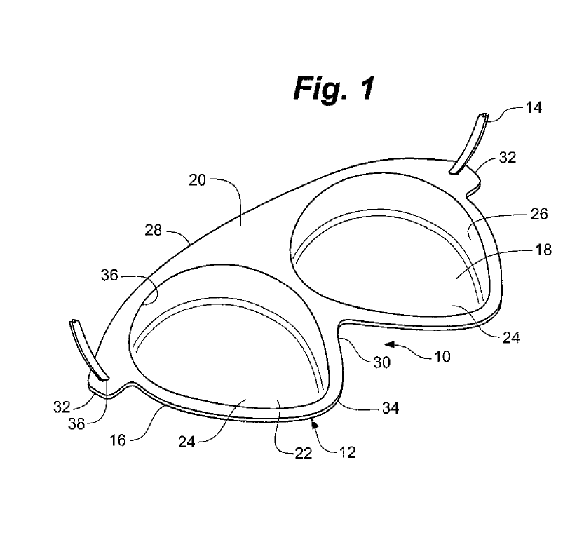

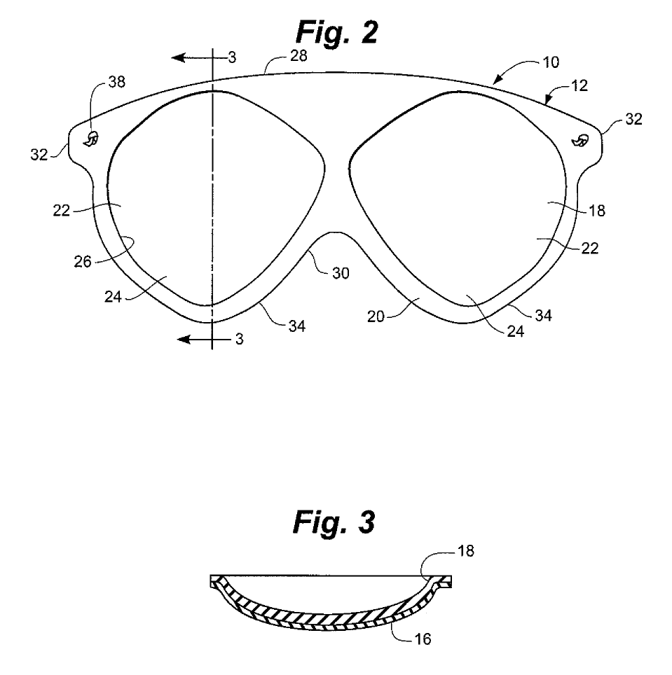

Referring to FIG. 1, in one embodiment eye mask 10 of the invention generally includes shell 12 and retainer 14. Shell 12 generally includes substantially impermeable layer 16 and absorbent layer 18. Referring particularly to FIGS. 1 and 2, shell 12 generally presents eye chambers 22. Eye chambers 22 are defined by domes 24 and walls 26.

In this example embodiment, planar portion 20 generally defines brow arch 28, nose relief 30, retainer ears 32, cheek arches 34 and dome perimeters 36. Brow arch 28 is gently curved and stretches generally between retainer ears 32. Nose relief 30, is centrally located on the lower edge of shell 12 between eye chambers 22 and is formed by the juncture of check arches 34. Retainer ears 32 may optionally include apertures 38 for receiving retainers 14 therein. Retainers 14 may also be secured by for example stitching, stapling, fasteners, adhesives or other techniques known to the art.

Domes 24 and walls 26 together define eye chambers 22. Eye chambers 22 may take the shape of roughly an irregular pentagon as depicted, but this shape should not be considered to be in any way limiting. In addition, planar portion 20 is described as planar only when eye mask 10 is molded, because eye mask 10 is formed of flexible resilient material, planar portion 20 does not retain a planar configuration at all times such as when mask 10 is flexed.

Referring particularly to FIG. 3, substantially impermeable layer 16 and absorbent layer 18 are, in an example embodiment, tightly bonded to one another. As can be seen in planar portion 20, substantially impermeable layer 16 and absorbent layer 18 are relatively thin and compressed against one another. As can also be seen in the example depicted in FIG. 3, in the center of eye chambers 22, absorbent layer 18 is substantially thicker than substantially impermeable layer 16. In an example embodiment, absorbent layer 18 may be approximately 3 to 5 millimeters thick near the center of eye chambers 22 and approximately 1 to 2 millimeters thick near planar portion 20.

In an example embodiment of the invention substantially impermeable layer 16 has a generally consistent thickness throughout eye mask 10.

In an example embodiment of the invention, absorbent layer 18 may be formed of an open cell hydrophilic foam. In one example embodiment of the invention, the hydrophilic foam may be Medisponge 30W SOO-T or SOS-T produced by Lendell Manufacturing, Inc. Medisponge is a hydrophilic flexible polyurethane foam. In accordance with the invention, an example absorbent layer 18 may have the following properties.

| Property | Specification |
|---|---|
| Pore Structure | Uniform tight pore structure across width |
| Density, lb/ft³ | 5.7 +/− 1 lbs/ft³ |
| Tensile (psi) | 30.2 +/− 5 |
| % Volume Swell | 75% =/− 15% |
| Wet out | less than one minute |
| Saturated Water Retention | 15 +/− 4 times Wt/wt |
| Wrung Water Retention | 1.5 +/− 0.5 |

Substantially impermeable layer 16 in one example embodiment may be formed of a closed cell polyurethane foam. For example, polyethylene foam sheets, such as those that can be obtained from Voltek of Lawrence, Mass. have been found to be suitable for use in the present invention. The following examples illustrate suitable qualities.

### EXAMPLE 1

foam sheet thickness 4.0 mm (Voltek of Lawrence, Mass., Code: LL1077);

density 0.035 grams per cc;

water vapor transmission rate 0.087 g/8hours/100 sq inches-ASTM E96;

compression set 6% original thickness-ASTM D3575;

shore hardness 18 “A” scale; and

25% compression load 2.5 psi-ASTM D3575.

Such foam sheet material can be molded into a mask by heat and pressure. In this example the temperature of a mold was maintained at about 120°°C. and the molding time was approximately 2.5 minutes.

### EXAMPLE 2

Polyethylene foam sheets (Voltek Code: LL1036):

Foam sheet thickness 3.4 mm;

Density 0.21 grams cm³;

Water vapor transition rate 0.087 g/8 hours/100 sq inches-ASTM E96;

Compression set 3.8% original thickness;

Shore hardness 30 “A” scale; and

25% compression load 8.5 psi.

A mask produced from this material is less flexible than that described above in Example 1.

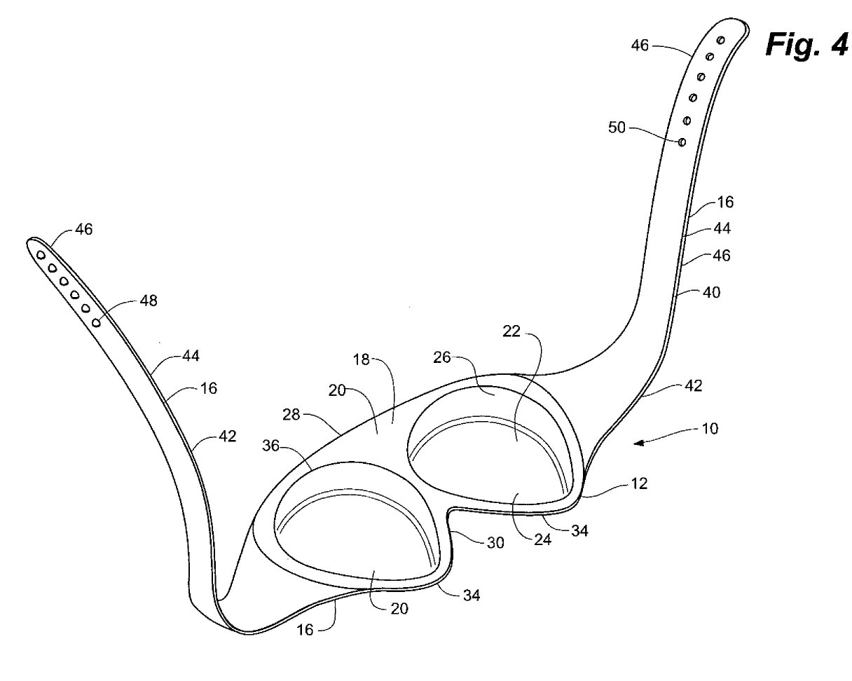

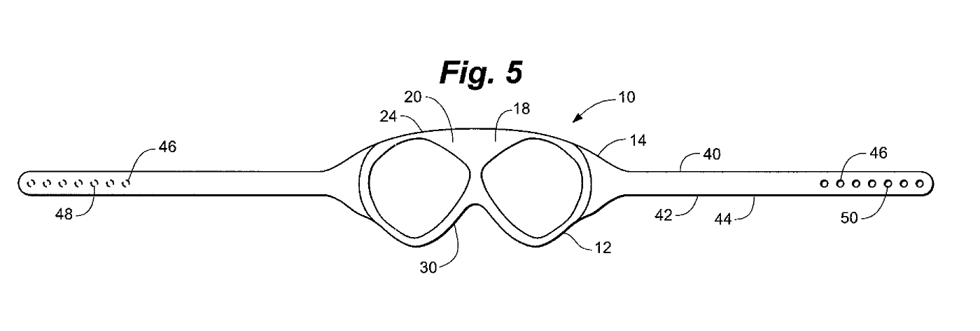

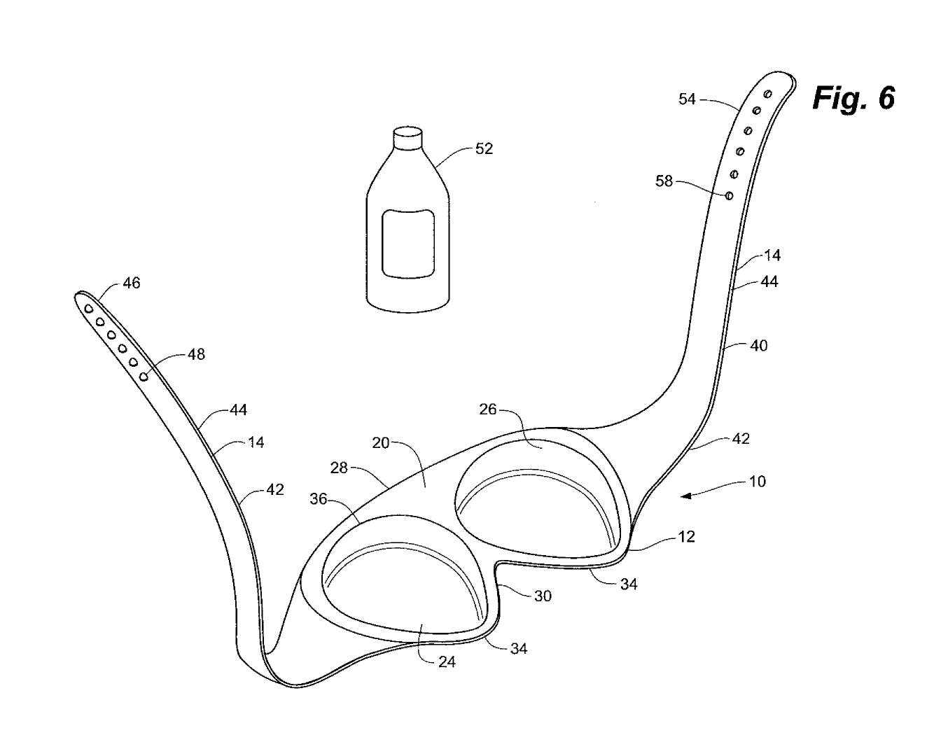

Referring to FIGS. 4-6, another example embodiment of the invention is depicted. In this example embodiment, eye mask 10 generally includes planar portion 20 and eye chambers 22 similar to those described above, but also includes integral retainers 40.

Integral retainers 40 may include extensions 42, straps 44 and coupling members 46. In this example, coupling members 46 include buttons 48 and apertures 50. Buttons 48 may be integrally molded to straps 44 and protrude therefrom. Buttons 48 may also be coupled in another way to straps 44. Apertures 50 are sized to receive buttons 48 with some degree of resistance so that buttons 48 may be forced into apertures 50 and retain in apertures 50. Coupling members 46 may also include hook and loop fasteners such as Velcro® fasteners, hooks and eyes as well as other techniques known to the art to couple straps 44 together.

Retainer 14 may also include an elastic member or hook and loop fasteners to secure eye mask 10 to the head of a user.

The invention may also include a kit containing eye mask 10 and an eye care solution 52 for moistening absorbent layer 18. Eye care solution 52 may include sterile water, sterile saline solution, an artificial tear solution or other solutions including medications appropriate for the treatment of dry eye syndrome.

In another example embodiment, the eye mask 10 may be provided in a sealed container pre-moisten with an eye care solution 52.

In a further example embodiment, the invention includes a kit including eye mask 10, eye care solution 52 and instructions to moisten absorbent layer 18 of eye mask 10 with eye care solution 52, to apply eye mask 10 over the eyes, and to maintain eye mask 10 over the eyes for a length of time to provide a beneficial therapeutic result. Eye mask 10 can also be moistened with plain water in lieu of eye care solution 52 and may be used during sleep or during any period of time desired while resting the eyes.

In an example embodiment, eye care solution 52 can be an aqueous solution containing electrolytes. For example, a formulation may include sterile water 98.9%, sodium chloride 0.5%, potassium chloride 0.2% and glycerin 0.4%. In this example of eye care solution 52, glycerin acts as a lubricant and controls the rate of moisture evaporation.

In an example embodiment, for distribution purposes, eye mask 10 and eye care solution 52 can be packaged in a conventional plastic container formed from, for example, polyester or polyuthrene. The packaging may be sealed airtight to preclude drying if eye mask is pre-moistened and to maintain sterility.

In an example embodiment, eye mask 10 can be sterilized by application of Gamma radiation. Approximately a 20-30 kilogray radiation dosage is expected to be required to sterilize eye mask 10.

The invention also includes a method of making an eye mask 10 for the relief of dry eye in a dry eye subject. The method includes molding unitary shell 12 shaped to cover at least one eye and a portion of a face of the dry eye subject. Substantially impermeable layer 16 is selected to be substantially moisture impermeable. For example, polyethylene foam as described above may be used. Absorbent layer 18 may be formed from, for example polyurethane foam as described above. Substantially impermeable layer 16 overlaid over absorbent layer 18 and the layers are secured to each other at a layer interface. Substantially impermeable layer 16 and absorbent layer 18 are substantially coextensive over their entire interface. Substantially impermeable layer 16 and absorbent layer 18 may be secured to each other by thermoforming or by use of adhesives.

In operation, eye mask 10 is supplied in a pre-moistened state or dry with a supply of eye care solution 52. A user removes eye mask 10 from the container and if pre-moistened applies it over the eyes. If eye mask 10 is not pre-moistened, the user applies eye care solution 52 to eye mask 10 and applies eye mask 10 over the eyes. The user may also moisten eye mask ten with plain water and wring out excess water. While over the eyes, eye mask 10 creates and maintains a humidity rich environment to provide improved comfort in eye health to a dry eye sufferer.

The invention may be embodied in other specific forms without departing from the spirit of the essential attributes thereof therefore, the illustrated embodiments should be considered in all respects as illustrative and not restrictive, reference being made to the appended claims rather than to the forgoing description to indicate the scope of the invention.

1. A mask for the relief of dry eye in a dry eye subject, comprising:
a unitary shell shaped to cover at least one eye and a portion of a face of the dry eye subject, the shell comprising a first layer and a second layer the first layer being a substantially moisture impermeable layer and the second layer being a moisture absorbent layer;
the first layer and the second layer meeting at a layer interface; and
the first layer and the second layer being substantially coextensive over substantially an entirety of a layer interface and secured together over substantially the entirety of the layer interface.

2. The mask as claimed in claim 1, wherein the first layer and the second layer are fused together.

3. The mask as claimed in claim 1, wherein the first layer and the second layer are bonded together.

4. The mask as claimed in claim 1, further comprising an integral retaining member that is contiguous with at least the first layer.

5. The mask as claimed in claim 1, wherein the second layer demonstrates a saturated water retention of fifteen times a weight of the second layer plus or minus about four times the weight of the second layer on weight to weight basis.

6. The mask as claimed in claim 1, further comprising an eye care solution absorbed by the second layer.

7. A method of making a mask for the relief of dry eye in a dry eye subject, comprising:
molding a unitary shell shaped to cover at least one eye and a portion of a face of the dry eye subject;
selecting a first layer and a second layer, the first layer being a substantially moisture impermeable layer and the second layer being a moisture absorbent layer;
overlaying the first layer and the second layer to meet at a layer interface;
placing the first layer and the second layer into contact substantially coextensively over substantially an entirety of a layer interface; and
securing together the first layer and the second layer substantially over the entirety of the layer interface.

8. The method as claimed in claim 8, further comprising fusing the first layer and the second layer together.

9. The method as claimed in claim 7, further comprising applying heat and pressure to the first layer and the second layer in a mold.

10. The method as claimed in claim 7, further comprising bonding the first layer and the second layer together.

11. The method as claimed in claim 10, further comprising applying pressure to the first layer and the second layer in mold.

12. The method as claimed in claim 7, further comprising forming the mask to include an integral retaining member that is contiguous with at least the first layer.

13. The method as claimed in claim 7, further comprising selecting the second layer material such that the second layer material demonstrates a saturated water retention of fifteen times a weight of the second layer plus or minus about four times the weight of the second layer on weight to weight basis.

14. The method as claimed in claim 7, further comprising applying an eye care solution to be absorbed by the second layer.

15. The method as claimed in claim 14, further comprising packaging the mask that has been moistened with eye care solution in a sealed packaging.

16. A mask for the relief of dry eye in a dry eye subject, comprising:
means for covering at least one eye and a portion of a face of the dry eye subject, the means for covering comprising a first layer and a second layer the first layer being formed from means for inhibiting the passage of moisture and the second layer being formed from means for absorbing moisture;
the first layer and the second layer meeting at a layer interface;
the first layer and the second layer being substantially coextensive over substantially an entirety of a layer interface and secured together over substantially the entirety of the layer interface.

17. The mask as claimed in claim 16, wherein the first layer and the second layer are fused together.

18. The mask as claimed in claim 16, wherein the first layer and the second layer are bonded together.

19. The mask as claimed in claim 16, further comprising means for retaining the mask on the face of the dry eye subject that is contiguous with at least the first layer.

20. The mask as claimed in claim 16, wherein the means for absorbing moisture demonstrates a saturated water retention of fifteen times a weight of the means for absorbing moisture plus or minus about four time s the weight of the means for absorbing moisture on weight to weight basis.

21. The mask as claimed in claim 16, further comprising an eye care solution absorbed by the means for absorbing moisture.
